(19) United States
(12) Patent Application Publication
(10) Pub. No.: US 2011/0093205 A1
(43) Pub. Date: Apr. 21, 2011

(54) PROTEOMICS PREVIEWER

(75) Inventor: Marshall W. Bern, San Carlos, CA (US)

(73) Assignee: Palo Alto Research Center Incorporated, Palo Alto, CA (US)

(21) Appl. No.: 12/581,503

(22) Filed: Oct. 19, 2009

(51) Int. Cl. G06F 19/00; G01N 33/48; G06F 17/18

(52) U.S. Cl. 702/19; 702/179

(57) ABSTRACT

A technique for analyzing proteomics data (such as tandem mass-spectrometry data) corresponding to peptides in a sample is described. In a high-speed, low-sensitivity first pass of this analysis technique, analysis parameters, such as the presence of one or more potential modifications to the one or more peptides, are determined using a representative subset of a database of known proteins. For example, a given potential modification in the one or more potential modifications may be determined by comparing matches between measured mass-spectrometry spectra and generated theoretical mass-spectrometry spectra without the given potential modification to matches between the measured mass-spectrometry spectra and generated theoretical mass-spectrometry spectra with the given potential modification. Then, in a lower-speed, higher-sensitivity second pass of the analysis technique, one or more peptides and/or proteins in the proteomics data are identified using the database of known proteins and the determined analysis parameters.

### BACKGROUND

1. Field of the Invention

The present invention relates to techniques for analyzing mass-spectrometry data. More specifically, the present invention relates to the analysis of mass-spectrometry data for peptides.

2. Related Art

In proteomics, proteins are often identified using mass spectrometry. A protein sample is typically digested into peptides that include one or more amino acids. For example, the protein sample can be digested using the enzyme trypsin. The resulting peptides can be ionized using matrix-assisted laser-desorption ionization or electro-spray ionization and introduced into a mass spectrometer. Tandem mass spectrometry measures the mass-to-charge ratios of the peptides, and then fragments the peptides and measures the mass-to-charge ratio of the resulting fragments. Peptide identifications made from tandem-mass-spectrometry data can be aggregated to identify the proteins in the sample.

In principle, the peptides in the sample can be uniquely identified using the peaks in the resulting mass-spectrometry spectra (which are associated with the mass-to-charge ratios of the peptides and peptide fragments). For example, peptides may be identified by comparing the observed mass-spectrometry spectra to theoretical mass-spectrometry spectra of peptides predicted by gene sequences or to previously observed mass-spectrometry spectra for known peptides.

In practice, however, it is often difficult to identify the peptides. For example, there may be chemical modifications to the amino acids in the peptides. These chemical modifications may be in vivo post-translational modifications or simply chemical artifacts, such as modifications that occur when the protein sample is prepared for mass-spectrometry analysis. When present, the chemical modifications can lead to shifts in the peaks in the mass-spectrometry spectrum of a peptide, which can complicate or confound the identification of the peptide based on comparisons with the previously observed or theoretically predicted mass-spectrometry spectra for known peptides.

One existing analysis technique attempts to address this problem by shifting some or all of the peaks in the previously observed or theoretically predicted mass-spectrometry spectra, based on one or more chemical modifications that are anticipated (prior to the mass-spectrometry analysis) to occur in the protein sample. The mass-spectrometry spectra with shifted peaks can then be compared with the observed unknown mass-spectrometry spectrum in order to make an identification. Unfortunately, the chemical modifications in a protein sample are difficult to guess a priori. Moreover, there are more than 200 types of potential chemical modifications, and ten or more of these types may be present in a single protein sample, so it is often too computationally expensive to search for all combinations of all potential modifications. Consequently, this existing analysis technique may be too restrictive to properly analyze the observed mass-spectrometry spectra.

Another existing analysis technique uses a so-called “blind modification search” to identify the peptide represented in an observed mass-spectrometry spectrum. In this existing analysis technique, peaks in the observed mass-spectrometry spectrum are fit without using any prior knowledge of likely mass shifts, apart from upper and lower bounds on the size of the shift. Blind modification search, however, is often too general because it does not take advantage of chemical knowledge, such as the propensity of methionine to oxidize, or the likelihood of chemical artifacts at the peptide N-terminus.

Because of these problems with existing analysis techniques, in current proteomics studies a researcher typically guesses what type of search to perform in order to identify the peptides represented in an observed mass-spectrometry spectrum. A wide search may result in more identifications, but it will take longer. Moreover, an overly wide search usually has lower specificity, which may make it harder to distinguish correct identifications from false positives. Consequently, most researchers usually run one or two searches with a few reasonable settings or parameters, for example, a fully tryptic search with a few common modifications enabled, or a semi-tryptic search with no modifications enabled. Then, they pick the best result. However, these existing approaches are problematic because researchers do not know if the parameters used resulted in an optimal search.

Hence, what is needed is a method and an apparatus that facilitates analysis of mass-spectrometry data for peptides or proteins without the problems listed above.

### SUMMARY

One embodiment of the present invention provides a computer system to analyze proteomics data corresponding to peptides in a sample. During operation, the computer system determines analysis parameters during a first-pass analysis of the proteomics data using a representative subset of a database of known proteins. Then, the computer system identifies one or more peptides and/or proteins in the proteomics data in a second-pass analysis of the proteomics data using the database of known proteins and the determined analysis parameters.

In some embodiments, the proteomics data includes tandem mass-spectrometry data, which includes measured mass-spectrometry peak locations for fragments of the peptides and a corresponding measured total mass of the fragments of the peptides.

Furthermore, the analysis parameters may include: mass errors associated with the mass-spectrometry peak locations; one or more quality metrics associated with quality of the sample; one or more quality metrics associated with preparation of the sample; one or more quality metrics associated with performance of a mass spectrometer that measures the mass-spectrometry peak locations for the fragments of the peptides and the corresponding measured total mass of the fragments of the peptides; and/or one or more potential modifications to one or more of the peptides. For example, the one or more potential modifications may include: methylation, dimethylation, oxidation (such as oxidized methionine), deamidation, carbamylation, phosphorylation or acetylation, as well as deliberate chemical treatments such as isotope labeling, lysine, cysteine and/or N-terminal modifications.

In some embodiments, the prevalence of a given potential modification is determined statistically. For example, the prevalence of a given potential modification may be determined by comparing the number of matches between the measured mass-spectrometry spectra (which include the mass-spectrometry peak locations) and generated theoretical mass-spectrometry spectra without the given potential modification to the number of matches between the measured mass-spectrometry spectra and generated theoretical mass-spectrometry spectra with the given potential modification. The generated theoretical mass-spectrometry spectra with the given potential modification may include permutations and combinations of the given potential modification at one or more amino-acid residues in the peptides.

Note that the representative subset of the database of known proteins may be based on a predefined group of potential modifications to the peptides, and the one or more potential modifications may be included in the predefined group of potential modifications. Furthermore, note that the first-pass analysis may be faster than the second-pass analysis, but may have a reduced sensitivity for identifying proteins in the proteomics data. Additionally, note that many potential modifications apply to many of the peptides in proteins in the sample, regardless of which protein the peptides came from. Consequently, the first-pass analysis may not need high protein sensitivity to determine the prevalence of potential modifications.

Another embodiment provides a method including at least some of the above-described operations.

Another embodiment provides a computer-program product for use in conjunction with the computer system.

### BRIEF DESCRIPTION OF THE FIGURES

FIG. 1A is a graph illustrating a tandem-mass-spectrometry spectrum in accordance with an embodiment of the present invention.

FIG. 1B is a graph illustrating a tandem-mass-spectrometry spectrum in accordance with an embodiment of the present invention.

FIG. 2 is a flow chart illustrating a process for analyzing proteomics data corresponding to peptides in a sample in accordance with an embodiment of the present invention.

FIG. 3 is a block diagram of a user interface during multi-pass analysis of proteomics data in accordance with an embodiment of the present invention.

FIG. 4 is a block diagram illustrating a computer system in accordance with an embodiment of the present invention.

FIG. 5 is a block diagram illustrating a data structure in accordance with an embodiment of the present invention.

Note that like reference numerals refer to corresponding parts throughout the drawings. Moreover, multiple instances of the same part are designated by a common prefix separated from an instance number by a dash.

### DETAILED DESCRIPTION

The following description is presented to enable any person skilled in the art to make and use the invention, and is provided in the context of a particular application and its requirements. Various modifications to the disclosed embodiments will be readily apparent to those skilled in the art, and the general principles defined herein may be applied to other embodiments and applications without departing from the spirit and scope of the present invention. Thus, the present invention is not intended to be limited to the embodiments shown, but is to be accorded the widest scope consistent with the principles and features disclosed herein.

Embodiments of a computer system, a method, and a computer-program product (e.g., software) for analyzing proteomics data (such as mass-spectrometry data) corresponding to peptides in a sample are described. In a high-speed, low-sensitivity first pass of this analysis technique, analysis parameters, such as the presence of one or more potential modifications to the one or more peptides, are determined using a representative subset of a database of known proteins. For example, a given potential modification in the one or more potential modifications may be determined by comparing matches between measured mass-spectrometry spectra and generated theoretical mass-spectrometry spectra without the given potential modification to matches between the measured mass-spectrometry spectra and generated theoretical mass-spectrometry spectra with the given potential modification. Then, in a lower-speed, higher-sensitivity second pass of the analysis technique, one or more peptides and/or proteins in the proteomics data are identified using the database of known proteins and the determined analysis parameters.

By using a multi-pass approach, this analysis technique can reduce the time and cost associated with the analysis of mass-spectrometry data. In particular, the analysis technique can increase the search speed, and can improve the quality of the analysis results.

We now discuss embodiments of a multi-pass analysis technique, including a first pass (which is sometimes referred to as a ‘previewer’) that identifies analysis parameters for use in a second pass that identifies one or more peptides and/or proteins based on mass-spectrometry data. During tandem mass spectrometry, a fragmentation spectrum is generated. This spectrum includes spectral peaks corresponding to fragments of a precursor (or “parent”) ion, which includes molecular subunits that are connected at cleavage sites. In particular, in a first stage of the tandem mass spectrometer, charged molecules (the parent ions) that have approximately the same ratio of mass-to-charge (m/z) are selected (typically, within a narrow tolerance). Then, in a second stage of the tandem mass spectrometer, the selected parent ions are fragmented at cleavage sites. These fragments are accumulated in m/z histogram bins. A number of these bins can represent a single spectral peak in a mass-spectrometry spectrum. Moreover, the number of counts in a given spectral peak (i.e., the height), the area under the given spectral peak, or a combination of the height and area of the spectral peak can be used to calculate the intensity of the given spectral peak. Note that the charge z for the fragments of the parent ion is typically 1, so that the position along the x-axis in the mass-spectrometry spectrum corresponds directly to mass for most peaks.

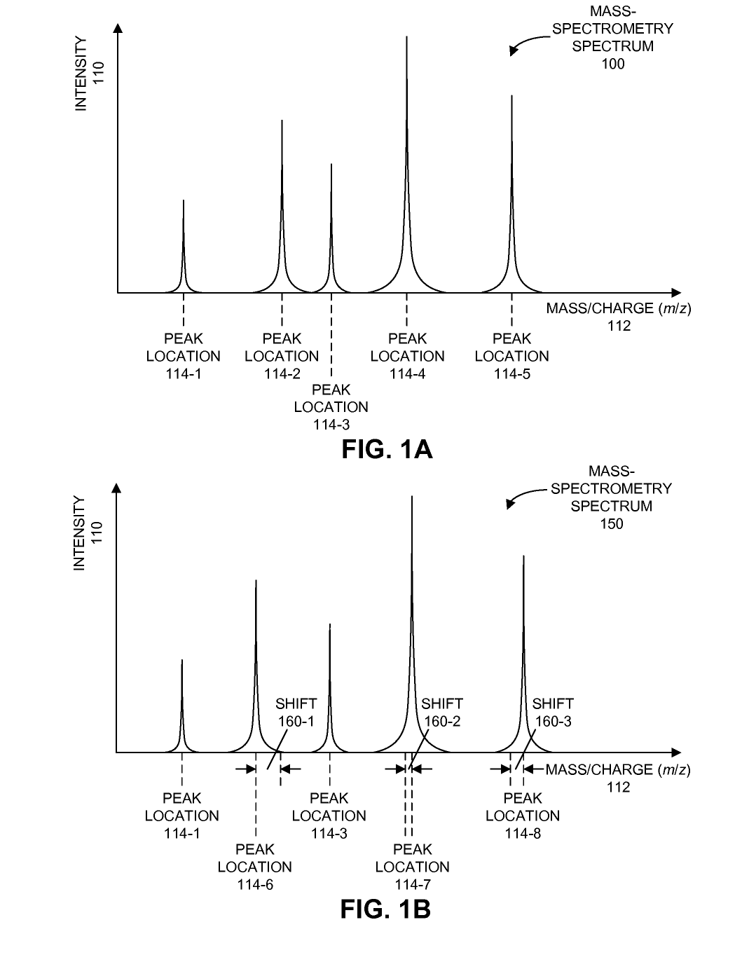

FIG. 1A presents a graph illustrating a mass-spectrometry spectrum 100 of a parent ion plotted as intensity 110 as a function of m/z 112. In this mass-spectrometry spectrum, peaks at peak locations 114 are associated with fragments of the parent ion(s) that have specific masses. In some embodiments, the parent ion is: a protein, a peptide (i.e., a portion of a protein), a lipid, a polymer (which is composed of multiple monomers), a glycan, and/or another organic compound or molecule.

For example, the parent ion may be a peptide and the fragments of the parent ion are smaller peptides. The parent peptide includes a sequence of amino-acid residues that are connected by peptide bonds, which are likely cleavage sites. A pair of fragments that are dissociated by a tandem mass spectrometer may be created by breaking the parent peptide at a given cleavage site. Thus, if the peptide includes the amino-acid sequence alanine-methionine-cysteine-aspartic acid-glutamic acid (AMCDE), the fragments may include: A, MCDE, AM, CDE, AMC, DE, AMCD, and/or E. Moreover, the intensity 110 of the corresponding spectral peaks in mass-spectrometry spectrum 100 may indicate how often the parent ion(s) have been fragmented at a particular cleavage site.

For peptides and proteins, it can be difficult to identify a particular peptide or protein from the peak locations 114. One source of this difficulty is chemical modification of amino-acid residues in the peptide. As shown in FIG. 1B, which presents a graph illustrating a mass-spectrometry spectrum 150, these chemical modifications result in shifts 160 of some of the peak locations 114 (such as peak locations 114-6, 114-7 and 114-8) that are associated with affected amino acids.

For example, M in the peptide AMCDE may be oxidized, which results in a mass shift of all of the peak locations 114 that are associated with fragments that include M (i.e., the peak locations associated with MCDE, AM, AMC and/or AMCD, which include half of the fragments). These peaks may all be shifted by the same amount (+16 Daltons in the case of oxidized methionine), while the remainder of the peaks may be at their original theoretical peak locations.

If the chemical modification(s) of the amino acids in the protein sample are known (prior to the mass-spectrometry analysis), then software such as the peptide identification programs Mascot (from Matrix Science, Inc., of London, England) and SEQUEST (from Thermo Fisher Scientific, Inc., of Waltham, Mass.) can be instructed to search for peptides that include the chemical modification(s) and which match the peak locations 114 (which is referred to as a “known” modification search). In this way, modified peptides can be identified.

Unfortunately, the chemical modification(s) to the peptides or proteins in the sample are not all known in advance. Moreover, there are 100 or more possible types of chemical modifications (such as methylation, dimethylation, oxidation, deamidation, carbamylation, phosphorylation or acetylation), and as many as five to ten of these types may be present in a given protein sample. Note that the chemical modifications present typically depend on both the biological system and on the chemical processing of the protein sample. Some modifications such as oxidized methionine, pyro-glu transformation of N-terminal glutamine or aspartic acid, and deamidation of glutamine and asparagine are ubiquitous, found in almost every sample, whereas some other modifications are found only in certain samples. Searching for all the mass shifts that occur in a given sample without knowledge of the common chemical modifications (using a so-called “blind search”) is extremely time consuming and error-prone, and produces results that can be hard to interpret. (For more information on blind-modification search techniques, such as the Popitam method, see Hernandez et al., Proteomics, Vol. 3., No. 6, 2003, 870-878, or InsPecT, see Tsur et al., Nature Biotechnology, Vol. 23, 1562-1567.)

An intermediate approach between a known modification search and a blind search is a so-called “wild-card-modification” analysis or search technique, which considers a range of potential mass shifts (typically integer) to amino-acid residues in one or more fragments of a peptide or a protein. This type of search allows users to build in knowledge of the modifications which are already known or suspected to occur in the protein sample. Thus, the search does not waste time or make errors in discovering something that is already known, such as the propensity of methionine to oxidize. By more selectively choosing the potential chemical modifications considered per peptide, the wild-card-modification technique allows faster, cheaper and more accurate identification of the peptide or protein sample being analyzed than other search techniques. Moreover, candidate identifications using wild-card modifications can be compared directly with candidate identifications using only known modifications, so that the strength of evidence for the unknown modification can be assessed statistically.

Note that a wild-card modification can be enabled along with any combination of known modifications. In particular, the shift associated with a known chemical modification is distinct from the peak shift caused by an unknown modification, and a theoretical ion that includes both known and unknown modifications gives a theoretical peak at a location shifted by the sum of these two types of shifts. Moreover, note that the unknown modification is called a “wild-card modification,” because it can match any mass addition or subtraction (which may be an integer or a non-integer within a range of masses) at any location within the peptide.

For example, a wild-card-modification search may allow any integer mass shift on any one residue within each candidate peptide. More precisely, the considered mass additions or subtractions may be exactly those additions or subtractions of integer masses that, along with the assumed known modifications, yield a total mass for the candidate peptide within the considered precursor mass range. Moreover, the wild-card modification may be restricted to certain amino-acid residues or to the N-terminal or C-terminal residue within each peptide.

Alternatively, the wild-card-modification search may be designed for high-accuracy tandem mass spectrometry (such as Fourier-transform mass spectrometry or quadrupole time-of-flight mass spectrometry). In particular, the wild-card-modification search may allow mass additions and subtractions that are not an integer number of Daltons (atomic mass units). For example, if the precursor mass is known to be 1290.76+/−0.01 Daltons, and the candidate peptide EKAEGDAALNR without a wild-card modification has a theoretical mass of 1272.66 Daltons, then the wild-card-modification search may include only additions of 18.10 Daltons, that is, the candidates E[+18.10 Daltons]KAEGDAALNR, EK[+18.10 Daltons]AEGDAALNR, and so forth.

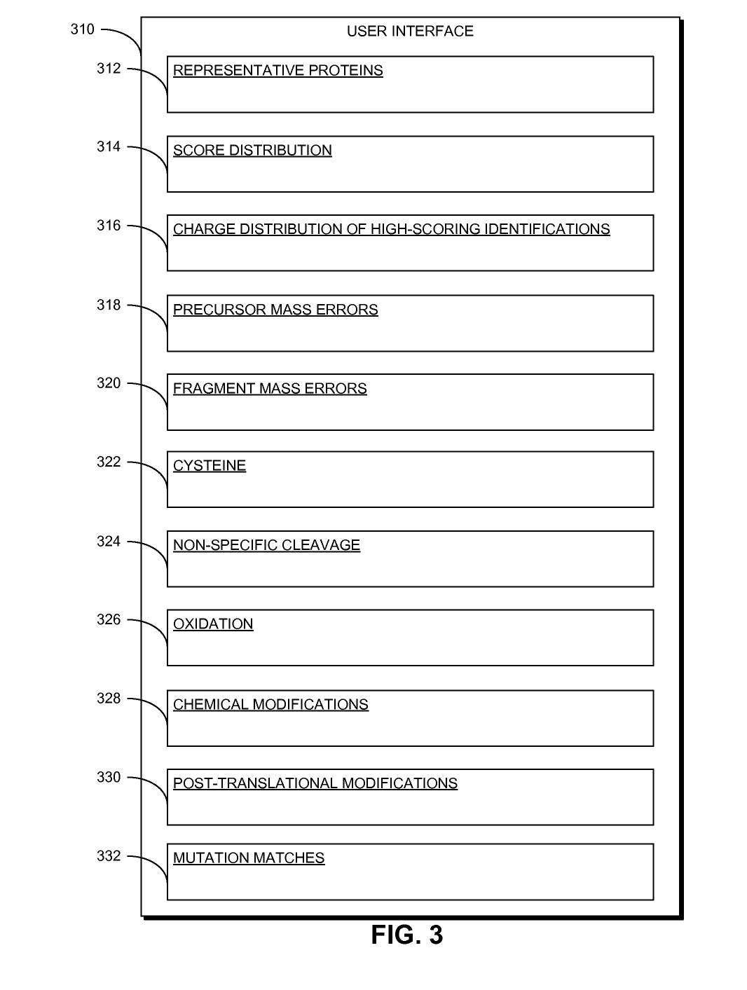

While wild-card-modification search is typically faster than blind search, it is still time consuming to perform, and may not provide an optimal search. In the discussion that follows, a very fast search tool (for example, up to 200× faster than existing search techniques) that is used during first-pass analysis is described. This fast search tool or previewer, in effect, tests hundreds of different searches, and provides a researcher with a recommendation for the “optimal” search. In the process, the fast search tool trades off speed and sensitivity. For example, it may assume that most modifications occur more or less uniformly across all the proteins in a sample. Based on this assumption, oxidation can be detected simply by checking the most abundant proteins. Thus, the search tool may first perform a fast narrow search with only a few modifications enabled, and may compile small databases of observed proteins and peptides for use in all subsequent searches by the search tool. As described further below with reference to FIG. 3, these subsequent searches may check for: nonspecific cleavage, common modifications (which may be considered one at a time), unanticipated (wild-card) N-terminal modifications, and unanticipated modifications at any residue. Furthermore, the search tool may gather and provide statistics, such as: an estimated of number of semi-tryptic peptides with ragged N-terminus, an estimated oxidation rate of methionines, etc. Additionally, the search tool may provide a list of the most abundant proteins (such as the ones used to gather the statistics) or any of the abundant proteins that are likely to be in the sample. (Thus, even though the search tool is not, per se, being used to identify proteins in a sample, it may provide some candidate proteins for use in subsequent analysis passes that are trying to identify proteins.) In some embodiments, the search tool provides m/z recalibration settings, i.e., measurement errors of the mass spectrometer.

After completing the first-pass analysis using the search tool, additional analysis passes may be performed using other search techniques. For example, a known search, a wild-card-modification search and/or a blind search may be performed to identify one or more peptides and/or proteins. In some embodiments, identifying the one or more peptides and/or proteins involves an iterative process, in which results from a previous iteration are used as ‘known’ results in a subsequent iteration. Moreover, the one or more additional analysis passes may leverage the analysis parameters determined in the first pass using the search tool. In this way, the search tool may assist a researcher in accurately and quickly identifying one or more peptides and/or proteins in a sample using an optimal (or near optimal) search based on the analysis parameters.

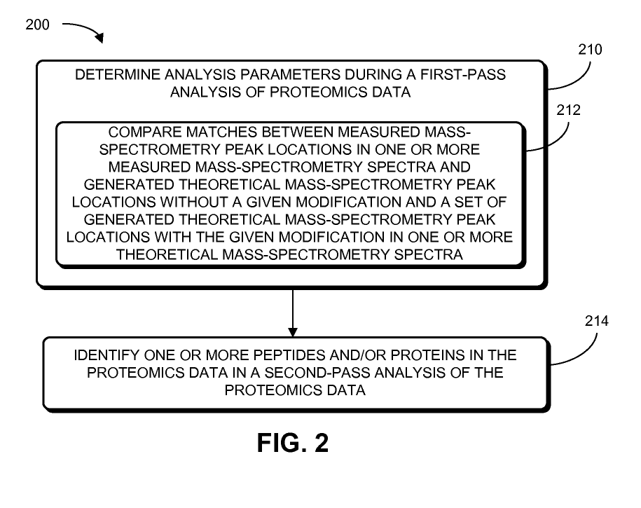

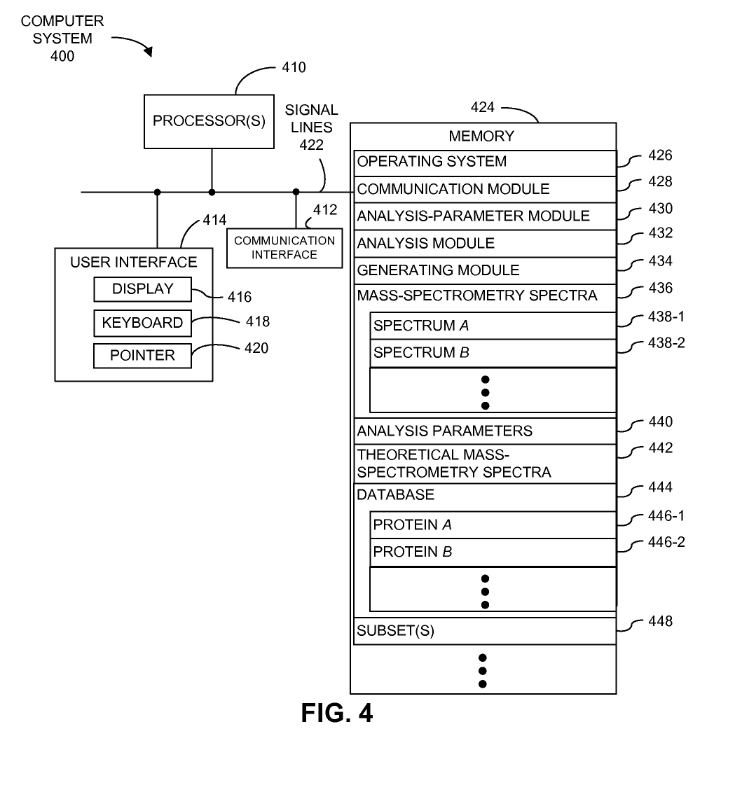

FIG. 2 presents a flow chart illustrating a process 200 for analyzing proteomics data corresponding to peptides in a sample. The process may be performed by a computer system (such as computer system 400 in FIG. 4). During operation, the computer system determines analysis parameters during a first-pass analysis of the proteomics data using a representative subset of a database of known proteins (operation 210). In some embodiments, the proteomics data includes mass-spectrometry data, which includes measured mass-spectrometry peak locations in one or more measured mass-spectrometry spectra (such as tandem mass-spectrometry spectra) for fragments of the peptides and a corresponding measured total mass of the fragments of the peptides.

Furthermore, the analysis parameters may include: mass errors associated with the mass-spectrometry peak locations; one or more quality metrics associated with quality of the sample; one or more quality metrics associated with preparation of the sample; one or more quality metrics associated with performance of a mass spectrometer that measures the mass-spectrometry peak locations for the fragments of the peptides and the corresponding measured total mass of the fragments of the peptides; and/or one or more potential modifications to one or more of the peptides. Note that the one or more potential modifications may include: methylation, dimethylation, oxidation (such as oxidized methionine), phosphorylation and/or acetylation, as well as deliberate chemical treatments such as isotope labeling, lysine, cysteine and/or N-terminal modifications.

Moreover, a given potential modification in the one or more potential modifications may be determined statistically, such as a number of peptide matches in the subset of the database when the given potential modification is included. For example, a given potential modification in the one or more potential modifications may be determined by comparing or evaluating matches between the measured mass-spectrometry peak locations across one or more measured mass-spectrometry spectra and generated theoretical mass-spectrometry peak locations without the given potential modification across one or more theoretical mass-spectrometry spectra to matches between the measured mass-spectrometry peak locations across the one or more measured mass-spectrometry spectra and generated theoretical mass-spectrometry peak locations with the given potential modification across the one or more theoretical mass-spectrometry spectra (operation 212). In an exemplary embodiment, the proteomics data includes 10,000 measured mass-spectrometry spectra, which each include 100 mass-spectrometry peak locations.

Note that the one or more theoretical mass-spectrometry spectra may include permutations and combinations of the given potential modification at one or more amino-acid residues in the peptides. Therefore, if a peptide includes two instances of an amino-acid residue, the generated theoretical mass-spectrometry peak locations may include mass-spectrometry peak locations for the given modification occurring at a first instance of the amino-acid residue, at a second instance of the amino-acid residue and/or at both instances of the amino-acid residue. Note that the potential modifications may be tried one at a time in successive passes over the proteomics data. Alternatively or additionally, a wild-card-modification search may be used to test the potential modifications.

Evaluating a candidate peptide identification for a tandem mass-spectrometry spectrum is called “peptide scoring.” Peptide scoring is familiar to anyone skilled in the art, but for completeness this process is described in more detail below. Some embodiments may evaluate an explanation by checking fragment peaks before the parent mass, or the parent mass before fragment peaks. Other embodiments may bring additional information into the evaluation of the explanation, for example, the identity of the protein(s) containing the candidate peptide.

Scoring of candidate peptides with one or more potential modifications (which are sometimes referred to as “explanations”) may be similar to the peptide scoring performed by existing software programs such as Mascot, SEQUEST, and X!Tandem (from the Global Proteome Machine Organization). For example, for each measured tandem mass-spectrometry spectrum, the computer may assemble: a list of candidate peptides, based upon the precursor mass associated with the tandem mass-spectrometry spectrum; the locations of peaks within the tandem mass-spectrometry spectrum; and/or a “sequence tag” (partial amino acid sequence) deduced de novo from the tandem mass-spectrometry spectrum, a priori knowledge of the protein sample, or any other information relevant to the selection of candidate peptides. Thus, the computer system may first compare the mass of an explanation with the observed precursor mass of the spectrum to be identified. The observed precursor mass may be derived from the observed precursor mass-over-charge using either an observed or presumed charge for the peptide. In most proteomics experiments, peptide charges are +1, +2, +3, or +4, so all four possibilities can be tried if the actual charge cannot be observed.

Then, the computer system may evaluate the explanation by generating a theoretical mass-spectrometry spectrum for the explanation. This theoretical mass-spectrometry spectrum may include peaks corresponding to expected ions. Note that expected ions are known to those skilled in the art, and include ions corresponding to prefixes and suffixes of the amino-acid residue sequence, such as a-, b-, and c-ions (three types of prefix ions) and y- and z-ions (two types of suffix ions). Expected ions may also include prefix and suffix ions modified by the loss of water or ammonia. A scoring function may take into account the number or fraction of theoretical ions matched (within some mass tolerance) by peaks in the observed spectrum. Moreover, it may also take into account the number or fraction of peaks in the observed spectrum matched (within some mass tolerance) by theoretical ions. Furthermore, it may also take into account: the intensities of observed peaks, the predicted intensities of theoretical ions, and/or the magnitudes of the mass errors (the difference between theoretical and observed mass-over-charge values).

Note that the program may accept as correct each explanation that is the highest-scoring explanation for its spectrum and which has a score exceeding some threshold. The number of accepted explanations may vary depending upon whether a potential modification is considered. This statistical information may be used to determine potential modifications that are likely to be present in the sample. For example, a given potential modification may be likely to be in the sample if more than 0.1, 1, 2, 5 and/or 10% of the matches include this potential modification.

In some embodiments, a correction factor is applied when identifying potential modifications based on the size of the search space. In particular, the number of matches (with and without the given potential modification) between measured mass-spectrometry mass spectra and theoretical mass-spectrometry mass spectra generated using a nonsensical database may be subtracted, respectively, from the number of first matches and the number of second matches.

Next, the computer system identifies one or more peptides and/or proteins in the proteomics data in a second-pass analysis of the proteomics data using the database of known proteins and the determined analysis parameters (operation 214). For example, the second-pass analysis may include any identified potential modifications and/or may include potential peptides or proteins found using the search tool (for example, a subset of the abundant proteins which matched the proteomics data) as known peptides or proteins in the second-pass analysis. Thus, the analysis parameters may be used to refine or focus subsequent searches through the database of known proteins while identifying the one or more peptides and/or proteins in the proteomics data.

In some embodiments, during the second-pass analysis post-scoring filters are applied to reject certain identifications, for example, based on other channels of information, such as chromatographic retention time. Spectrum identifications can then be integrated into protein identifications by: counting the number of peptide identifications for each protein, counting the total scores of all peptide identifications for each protein, using various other protein- or group-assembly techniques in software programs such as: ProteinProphet (from the Institute for Systems Biology in Seattle, Wash.), Scaffold (from Proteome Software, Inc., of Portland, Oreg.), and PROVALT (as described by Weatherly et al., in Mol. Cell. Proteomics, Vol. 4, p. 762, June 2005).

Note that the representative subset of the database of known proteins may be based on a predefined group of potential modifications to the peptides, and the one or more potential modification may be included in the predefined group of potential modifications. In particular, the database may include tandem-mass-spectrometry data, which includes precursor masses (masses of the unfragmented peptides) along with fragmentation spectra of known peptides or proteins. Moreover, the predefined group of potential modifications may include known (that is, anticipated) chemical modification(s). For example, at least some of the potential chemical modification(s) may be inferred based on how a protein sample was prepared (such as a cysteine treatment). Furthermore, the predefined group of potential modifications may include potential modifications to approximately 100 abundant proteins.

Using the predefined group of potential modifications, a subset of the database of known proteins may be selected. In particular, the subset of the database may be that portion of the database which is relevant for the predefined group of potential modifications. This may speed up the first-pass analysis by reducing the size of the relevant portion of the database from 50,000 protein sequences (the size of a typical human protein database) to 1000 protein sequences (roughly the maximum number of proteins that can be identified in a single mass-spectrometry experiment). Consequently, the first-pass analysis may be faster than the second-pass analysis, but may have a reduced sensitivity for identifying proteins in the proteomics data.

In some embodiments, process 200 includes additional or fewer operations. Moreover, the order of the operations may be changed, and/or two or more operations may be combined into a single operation.

We now describe embodiments of a user interface that reports results (such as analysis parameters) of the first-pass analysis using the search tool of a measured mass-spectrometry spectrum (or set of spectra) and a database of known proteins (as well as associated mass-spectrometry spectra). FIG. 3 is a block diagram of a user interface 310 during multi-pass analysis of proteomics data. This user interface may provide representative proteins 312 (such as the abundant proteins) that were used in the first-pass analysis and/or that were identified in the first-pass analysis. For example, representative proteins 312 may include: rankings, scoring-function values, odds values, identifiers and/or protein names.

Moreover, user interface 310 may include a score distribution 314 for the scoring-function values of the abundant proteins considered during the first-pass analysis. For example, score distribution 314 may indicate that there were: 330 matches above 32 and 430 matches above 20, with a top score for reverse of 19.

In some embodiments, user interface 310 provides information about mass-spectrometer performance. For example, charge-distribution of high-scoring identifications 316 may indicate how many peptide charges had values of +1, +2, +3, or +4, such as: 14 occurrences of +1, 364 occurrences of +2, 30 occurrences of +3, and 0 occurrences of +4.

In addition, precursor mass errors 318 and fragment mass errors 320 may be reported. For example, a median m/z error (observed-true) in the precursor mass error may be 0.164, with 240 high and 168 low. Similarly, a median m/z error in the top identified peaks (observed-true) for fragment mass errors may be −0.0038, with 480 high and 954 low.

Other information in user interface 310 may indicate how well a sample was prepared. For example, there may be information about an intention modification, such as that to cysteine 322. This information may include how many matches occurred at +57 Daltons (iodoacetamide), as well as artifacts (such as N-terminus+57 Daltons/+14 Daltons carbamidmethylated).

Furthermore, there may be information that indicates how well the sample was digested or cut into specific pieces by an enzyme, such as non-specific cleavage 324. For example, user interface 310 may indicate that 28 or 379 matches correspond to a missed cleavage with an internal K or R not followed by a P. Similar information may be provided for semitryptic peptides and/or non-tryptic peptides.

Additionally, there may be one or more metrics of sample quality, such as oxidation 326 (which is a modification that occurs in vivo and in vitro, and can indicate sample degradation). For example, user interface 310 may indicate that: oxidized methianine occurred in 0.0% of 77 (non-unique) peptides; doubly oxidized methianine (M+32 Daltons) occurred in 0.0% of 69 (non-unique) peptides; oxidized histidine and tryptophan occurred in 0.0% of 135 (non-unique) peptides; triply oxidized cysteine (C+32 Daltons) occurred in 0.0% of 14 (non-unique) peptides; and arginine oxidation (carbonylation) to glutamic semialdehyde (R+43 Daltons) occurred in 0.0% of 144 (non-unique) peptides.

User interface 310 may also indicate any identified potential chemical modifications 328 (many of which occur in vivo, but some of which may occur in vitro). For example, user interface 310 may indicate that: deamidated asparagine or glutamine occurred in 10.2% of 324 peptides; pyro-glu N-terminal Q, E or camC occurred in 5.3% of 57 peptides; sodiation occurred in 0.0% of 375 peptides; carbamylation (N-terminus+43 Daltons, RK+43 Daltons) occurred in 0.0% of 375 peptides; carbamylated methionine (M+43 Daltons) occurred in 0.0% of 57 peptides; and acetaldehyde (N-terminus+26 Daltons, HK+26 Daltons) occurred in 0.0% of 375 peptides. A peptide number may be given either as the number of non-unique peptides (that is, the same peptide found 3 times is counted 3 times), or as the number of unique peptides (the same peptide found 3 times is counted once). Note that this statistical information (either absolute or relative) regarding peptide matches with and without a potential modification can be used to determine which potential modifications to selectively turn on or to include in the second-pass analysis. As noted previously, the search tool may consider the potential modifications in the predefined group of potential modifications one at a time and/or may perform a wild-card-modification search.

In some embodiments, user interface 310 provides peptides or proteins that were identified during the first-pass analysis. For example, post-translation modifications 330 (which typically occur in vivo) may indicate that: hydroxyproline occurred in 0.7% of 149 (non-unique) peptides that contain P; phosphorylation occurred in 0.3% of 349 (non-unique) peptides that contain S, T or Y; beta-elimination occurred in 0.0% of 319 (non-unique) peptides that contain S or T; methylation occurred in 0.3% of 358 (non-unique) peptides that contain K, H, N or R; dimethylation occurred in 0.0% of 347 (non-unique) peptides that contain K or R; and acetylation (or guanidination or trimethylation) occurred in 0.0% of 181 (non-unique) peptides. Additionally, mutation matches 332 may indicate that mutations (mass differences greater than −8 Daltons, except for mass differences of 0, −1, +1 and +16 Daltons) occurred in 0.5% of 366 (non-unique) peptides.

We now describe embodiments of a computer system that performs process 200. FIG. 4 presents a block diagram illustrating a computer system 400. Computer system 400 includes one or more processors 410, a communication interface 412, a user interface 414, and one or more signal lines 422 coupling these components together. Note that the one or more processing units 410 may support parallel processing and/or multi-threaded operation, the communication interface 412 may have a persistent communication connection, and the one or more signal lines 422 may constitute a communication bus. Moreover, the user interface 414 may include: a display 416, a keyboard 418, and/or a pointer 420, such as a mouse.

Memory 424 in the computer system 400 may include volatile memory and/or non-volatile memory. More specifically, memory 424 may include: ROM, RAM, EPROM, EEPROM, flash, one or more smart cards, one or more magnetic disc storage devices, and/or one or more optical storage devices. Memory 424 may store an operating system 426 that includes procedures (or a set of instructions) for handling various basic system services for performing hardware-dependent tasks. In some embodiments, the operating system 426 is a real-time operating system. Memory 424 may also store communication procedures (or a set of instructions) in a communication module 428. These communication procedures may be used for communicating with one or more computers, devices and/or servers, including computers, devices and/or servers that are remotely located with respect to the computer system 400.

Memory 424 may also include multiple program modules (or sets of instructions), including: analysis-parameter module 430 (or a set of instructions), analysis module 432 (or a set of instructions) and/or generating module 434 (or a set of instructions). Note that one or more of these program modules (or sets of instructions) may constitute a computer-program mechanism.

Analysis-parameter module 430 may determine analysis parameters 440 in a first-pass analysis of mass-spectrometry spectra 436, such as spectrum A 438-1 and spectrum B 438-2. In particular, analysis parameters 440 may be determined by comparing mass-spectrometry spectra 436 with theoretical mass-spectrometry spectra 442, which are generated by generating module 434 using subsets 448 of a database 444 of known proteins (such as protein A 446-1 or protein B 446-2). Note that generating module 434 may generate theoretical mass-spectrometry spectra 442 based on one or more potential chemical modifications that may be included in the sample being analyzed. Furthermore, note that database 444 may include approximately 20,000,000 unmodified tryptic peptide combinations (assuming only peptides having a length of 10-30 amino acids).

Subsequently, analysis module 432 may identify one or more peptides and/or proteins by performing a search through database 444 based on analysis parameters 440. In particular, the one or more peptides and/or proteins may be identified by comparing mass-spectrometry spectra 436 with theoretical mass-spectrometry spectra 442, which are generated by generating module 434 using database 444. For example, identified potential modifications in analysis parameters 440 may be included when generating theoretical mass-spectrometry spectra 442. In addition, peptides associated with any of the abundant proteins that were tentatively identified using analysis-parameter module 430 may be included when generating theoretical mass-spectrometry spectra 442. This identification process may include a known search, a blind search and/or a wild-card-modification search. Moreover, the identified peptides or proteins may be those peptides or proteins from the database 444 with the best matches to the observed spectra 436.

Instructions in the various modules in memory 424 may be implemented in: a high-level procedural language, an object-oriented programming language, and/or in an assembly or machine language. This programming language may be compiled or interpreted, i.e., configurable or configured, to be executed by the one or more processing units 410.

Although computer system 400 is illustrated as having a number of discrete items, FIG. 4 is intended to be a functional description of the various features that may be present in computer system 400 rather than a structural schematic of the embodiments described herein. In practice, and as recognized by those of ordinary skill in the art, the functions of computer system 400 may be distributed over a large number of devices or computers, with various groups of the devices or computers performing particular subsets of the functions. In some embodiments, some or all of the functionality of computer system 400 may be implemented in one or more application-specific integrated circuits (ASICs) and/or one or more digital signal processors (DSPs).

In some embodiments, computer system 400 includes fewer or additional components. Moreover, two or more components may be combined into a single component, and/or a position of one or more components may be changed. Moreover, the functionality of computer system 400 may be implemented more in hardware and less in software, or less in hardware and more in software, as is known in the art.

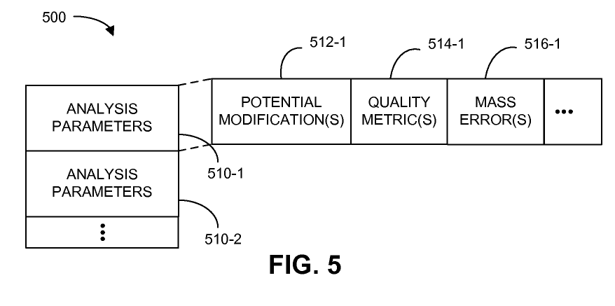

We now discuss data structures that may be used in computer system 400. FIG. 5 presents a block diagram illustrating a data structure 500. This data structure may contain analysis parameters 510. For example, analysis parameters 510-1 may include: one or more potential modifications 512-1, one or more quality metrics 514-1, and/or one or more mass errors 516-1.

In some embodiments, data structure 500 includes fewer or additional components. Moreover, two or more components may be combined into a single component and/or a position of one or more components may be changed.

While the preceding embodiments illustrate the use of the analysis technique in determining analysis parameters, such as one or more potential chemical modifications to peptides associated with a protein, in other embodiments the analysis technique may be used to analyze mass-spectrometry data associated with a wide variety of materials and chemical compounds, such as macromolecules that are made up of molecular subunits which are bound together at cleavage sites.

The foregoing descriptions of embodiments of the present invention have been presented for purposes of illustration and description only. They are not intended to be exhaustive or to limit the present invention to the forms disclosed. Accordingly, many modifications and variations will be apparent to practitioners skilled in the art. Additionally, the above disclosure is not intended to limit the present invention. The scope of the present invention is defined by the appended claims.

What is claimed is:

1. A method for analyzing proteomics data corresponding to peptides in a sample, comprising:
determining analysis parameters during a first-pass analysis of the proteomics data using a representative subset of a database of known proteins; and
identifying one or more peptides or proteins in the proteomics data in a second-pass analysis of the proteomics data using the database of known proteins and the determined analysis parameters.

2. The method of claim 1, wherein the proteomics data includes tandem mass-spectrometry data, which includes measured mass-spectrometry peak locations for fragments of the peptides and a corresponding measured total mass of the fragments of the peptides.

3. The method of claim 2, wherein the analysis parameters include mass errors associated with the mass-spectrometry peak locations.

4. The method of claim 2, wherein the analysis parameters include one or more potential modifications to one or more of the peptides.

5. The method of claim 4, wherein a given potential modification in the one or more potential modifications is determined statistically.

6. The method of claim 4, wherein a given potential modification in the one or more potential modifications is determined by comparing first matches between the measured mass-spectrometry peak locations and generated theoretical mass-spectrometry peak locations without the given potential modification to second matches between the measured mass-spectrometry peak locations and generated theoretical mass-spectrometry peak locations with the given potential modification.

7. The method of claim 6, wherein the generated theoretical mass-spectrometry peak locations with the given potential modification include permutations and combinations of the given potential modification at one or more amino-acid residues in the peptides.

8. The method of claim 4, wherein the representative subset of the database of known proteins is based on a predefined group of potential modifications to the peptides; and
wherein the one or more potential modifications are included in the predefined group of potential modifications.

9. The method of claim 4, wherein the one or more potential modifications include methylation, dimethylation, oxidation, deamidation, carbamylation, phosphorylation or acetylation.

10. The method of claim 2, wherein the analysis parameters include one or more quality metrics associated with performance of a mass spectrometer that measures the mass-spectrometry peak locations for the fragments of the peptides and the corresponding measured total mass of the fragments of the peptides.

11. The method of claim 1, wherein the analysis parameters include one or more quality metrics associated with preparation of the sample.

12. The method of claim 1, wherein the analysis parameters include one or more quality metrics associated with quality of the sample.

13. The method of claim 1, wherein the first-pass analysis is faster than the second-pass analysis; and
wherein the first-pass analysis has reduced sensitivity for identifying proteins in the proteomics data than the second-pass analysis.

14. A computer-program product for use in conjunction with a computer system, the computer-program product comprising a computer-readable storage medium and a computer-program mechanism embedded therein for analyzing proteomics data corresponding to peptides in a sample, the computer-program mechanism including:
instructions for determining analysis parameters during a first-pass analysis of the proteomics data using a representative subset of a database of known proteins; and
instructions for identifying one or more peptides or proteins in the proteomics data in a second-pass analysis of the proteomics data using the database of known proteins and the determined analysis parameters.

15. The computer-program product of claim 14, wherein the proteomics data includes tandem mass-spectrometry data, which includes measured mass-spectrometry peak locations for fragments of the peptides and a corresponding measured total mass of the fragments of the peptides.

16. The computer-program product of claim 15, wherein the analysis parameters include mass errors associated with the mass-spectrometry peak locations.

17. The computer-program product of claim 15, wherein the analysis parameters include one or more potential modifications to one or more of the peptides.

18. The computer-program product of claim 17, wherein a given potential modification in the one or more potential modifications is determined by comparing first matches between the measured mass-spectrometry peak locations and generated theoretical mass-spectrometry peak locations without the given potential modification and second matches between the measured mass-spectrometry peak locations to generated theoretical mass-spectrometry peak locations with the given potential modification.

19. The computer-program product of claim 18, wherein the generated theoretical mass-spectrometry peak locations with the given potential modification include permutations and combinations of the given potential modification at one or more amino-acid residues in the peptides.

20. The computer-program product of claim 17, wherein the representative subset of the database of known proteins is based on a predefined group of potential modifications to the peptides; and
wherein the one or more potential modifications are included in the predefined group of potential modifications.

21. The computer-program product of claim 15, wherein the analysis parameters include one or more quality metrics associated with performance of a mass spectrometer that measures the mass-spectrometry peak locations for the fragments of the peptides and the corresponding measured total mass of the fragments of the peptides.

22. The computer-program product of claim 14, wherein the analysis parameters include one or more quality metrics associated with preparation of the sample.

23. The computer-program product of claim 14, wherein the first-pass analysis is faster than the second-pass analysis; and
wherein the first-pass analysis has reduced sensitivity for identifying proteins in the proteomics data than the second-pass analysis.

24. A computer system, comprising:
a processor;
memory; and
a program module, wherein the program module is stored in the memory and configured to be executed by the processor to analyze proteomics data corresponding to peptides in a sample, the program module including:
instructions for determining analysis parameters during a first-pass analysis of the proteomics data using a representative subset of a database of known proteins; and
instructions for identifying one or more peptides or proteins in the proteomics data in a second-pass analysis of the proteomics data using the database of known proteins and the determined analysis parameters.
